(19) United States
(12) Patent Application Publication
(10) Pub. No.: US 2003/0167013 A1
(43) Pub. Date: Sep. 4, 2003

(54) Method and device of visualization of ECG-signals of standard leads with anatomic portrait of heart

(76) Inventors: Sula Anatoly, Moscow (RU); Vladimir Grishin, Mayfield Hts, OH (US); Kitashine Yuri, Moscow (RU); Reva Mikhail, Moscow (RU)

Correspondence Address: VLADIMIR GRISHIN, APT 845, 250 CHATHAM WAY, MAYFIELD HTS, OH, 44124, US

(21) Appl. No.: 10/358,419

(22) Filed: Feb. 4, 2003

Related U.S. Application Data
(60) Provisional application No. 60/353,336, filed on Feb. 4, 2002.

(51) Int. Cl. A61B 5/04

(52) U.S. Cl. 600/509

(57) ABSTRACT

This invention is in the medical area of electrocardiography (ECG) analysis. Despite its widespread use, well known that there is not enough sensitivity and specificity in many cases. The invention represents a radical improvement in ECG analysis in comparison to ECG analysis available today. This improvement in the diagnostic sensitivity and specificity to a wide array of serious and life-threatening cardiac abnormalities is made possible using standard ECG signals that are analyzed through the invented electrodynamical model of electrical field generation by the heart, which gives new information from ECG fluctuations unused by known methods based on static dipole model. It allows a much more detailed reconstruction of the heart's electrical processes and visualization of a precise anatomical picture of a dynamics of heart processes which heretofore has never been achieved. It allows the clinician to see whole pattern of heart state and to detect a wide range of cardiac abnormalities at a very early stage of disease and to identify these abnormalities (i.e., superior sensitivity and specificity when compared with modern ECG analysis).

### SPECIFICATION

This Utility Patent Application based on Provisional Patent Application of A. Soula at al. for “Method and Device of Visualization of ECG-signals of Standard Leads with Anatomic Portrait of Hearts”—

USPTO Serial No. 60/353,336, Filing date: Feb. 4, 2002, Docket No. GSN-20-5629

### CROSS-REFERENCE TO RELATED APPLICATIONS

Not Applicable

### STATEMENT REGARDING FEDERALLY SPONSORED RESEARCH OR DEVELOPMENT

Not Applicable—No Federal Sponsorship

### REFERENCE TO SEQUENCE LISTING, A TABLE, OR A COMPUTER PROGRAM LISTING

Not Applicable

### COMPACT DISK APPENDIX

Not Applicable

### BACKGROUND OF THE INVENTION

This invention is in the medical area of electrocardiography (ECG) analysis. ECG was first used in the beginning of the 20^{th} century. Despite its widespread use, clinicians acknowledge that there is not enough sensitivity and specificity in many cases when modern ECG analysis is used.

Because of this disadvantages of regular ECG methods and despite of common opinion that all fruitful information from ECG already has been got by means of standard PQRST wave properties, the authors developed new method which essentially surpass regular ECG analysis and has got European patent DE 198 01 240 A1 (Sula A. et al.: Verfahren und Vorrichtung zur Darstellung und Uberwachung von unktionsparametern eines physiologischen Systems, Jul. 29, 1999). The given patent describes a method of conversion of a signal one EKG lead in three-dimensional artificial topological model. The topological information of this model differs by that allow to extract the useful information from low amplitude fluctuations of the initial signal. This information is simply unavailable to traditional methods of the analysis, as these methods consider fluctuations, as narrow-band hum.

This property of topological models allows in 7 . . . 50 times to increase sensitivity and prognostic possibility of diagnostic devices. The main operations of the given method are:

a) the period of the registration of an ECG-signal is specified: practice it is 30-60 seconds practically,

b) inside this period the intervals of the analysis corresponding to all PQRST-complex are allocated. The number of such intervals is equal to number of PQRST-complexes in full record,

c) ECG signal inside each interval of the analysis is transformed in array of digital data, which are sent through a high-pass filter, than a difference between unfiltered and filtered signals is calculated, and a value of deviation between obtained data and empirical standard data of healthy hearts encode by color,

d) to each value of the obtained data array the space coordinates in three-dimensional space are assigned by the certain rules. These points build in three-dimensional space a frame (skeleton) of space figure called topological information model of the initial ECG signal.

e) through the obtained points of a frame in three-dimensional space of visualization the interpolating closed surface is carried out, color and shape of which directly depend on a state of a myocardium.

In outcome on the display screen the topological model of the initial ECG signal is built. On a way of construction it is information model of low amplitude fluctuations of an ECG-signal. The obtained in such a way map of fluctuations of an ECG signal as three-dimensional object is much more stable and sensitive than traditional features of ECG signal (PQRST complex). If to supervise dynamics of these topological models, it is possible to receive the very early and exact information on changes of a state of a myocardium. It allows essentially to increase sensitivity of the ECG analysis and to reliably detect starting pathological deviations even in a case, when on the regular ECG these change it are not found out yet.

However, this Method and Device of ECG Analysis has Two Principle Limitations:

At first, not all pathological states of heart appear in one lead. For example, small focal (transmural) myocardial infarction—in many cases it is impossible to reveal on one lead. The method—prototype does not give the exact rules how to use signals of multiple leads.

Secondly, at usage only one lead in a number of cases the various diseases have similar topological images that are indiscernible. It is connected that the information of one lead has not enough for finding of excitation direction and sharing of changes on the left and right atriums, left and right ventricles. Thereof, that method is effective only for highly sensitive detection of certain deviations, but is ineffective for derivation of diseases, i.e. exact definition of type of deviation and its localization. Besides there is no anatomical visualization as in this invention.

To remove these disadvantages, it is necessary from the analysis of fluctuations of one lead, i.e. analysis of electrical potentials of one point of body surface, to proceed to the analysis of fluctuations of electrical potentials of many surface points.

The method of such enhancement is given in patent DE 199 33 277 A1 (Soula A. at al, priority date—Jan. 20, 1999, open publication—Jan. 25, 2001). Further in the text the given method is named as “method—prototype”. This patent allows building of topological model with 12 standard ECG leads. The topological model of heart in this patent provides different color patterns for different diseases, i.e. allows effective diagnosis of disease type. However, the heart's topological model of patent DE 199 33 277 A1 has strictly discrete structure because the field of electrical fluctuations on body surface is formed only with 12 standard leads. As a result, surface points, which are not laying on lines of standard ECG leads, do not participate in forming of the topological model. Therefore, when surface potentials are constant in points of 12 standard leads but a little change in other points, the topological model does not show these changes. But they can be very important for detection of the earliest changes of fluctuation field preceding pathologies. For example, there is a wide group of clinically important hidden cases of ischemic disease, which are not showed by 12 standard leads of resting ECG but visible in other points. Therefore “the method—prototype” from patent DE 199 33 277 A1 is insensitive for this important diseases leading at unexpected clinical consequences.

### BRIEF SUMMARY OF THE INVENTION

The invention described herein represents a radical improvement in ECG analysis in comparison to ECG analysis available today. For standard EKG-signals from 6 or 12 standard EKG-leads the digital arrays of low amplitude fluctuations of EKG are defined and matched with similar array of an average PQRST-complex of PQRST-complexes selected during given time of exposure (up to 1 minute).

The obtained digital arrays of fluctuations are conversed into extended arrays of fluctuations of up to 180 additional EKG-leads by special algorithms. These algorithms are defined in this method on the basis of the principally new model of calculation of electromagnetic radiation of ionic currents in a myocardium, instead of calculation of only electrostatic fields of ionic currents, as it is accepted in other methods.

The extended arrays of fluctuations are a digital field, which is calculated for each point of an exterior surface of heart (epicardium). The amplitude of fluctuations in each calculated point depends on state of a myocardium near to this point.

The color-coding of amplitudes of fluctuations in the standard graphic palette is introduced: green color—norm, yellow—small deviations, red—expressed pathology. In outcome during exposure of standard EKG-signals the anatomic portrait of heart is formed, on which surface a fluctuation field immediately shows places and expressiveness of deviations. Each type of pathology has the specific color relief and appropriate heart portrait.

Supervising on the display screen obtained 3D the portrait it is possible more precisely to distinguish the different pathologies or their combinations. The fluctuation field varies much earlier, than there are deviations a standard ECG. Thereof this method provides drastical increasing both sensitivities, and specificity of the diagnosis, and also enables to supervise the earliest developments of pathology and its development, when other methods are not sensitive to such changes of a heart state. Besides, the fluctuation field is essentially more stable against the numerous disturbing factors than standard features of PQRST complex, that allows on the basis of proposed method to construct the effective automatic interpreter, much more precisely, than existing ones.

### BRIEF DESCRIPTION OF THE SEVERAL VIEWS OF THE DRAWING

FIG. 1 Approximate view of body surface potentials' map at the moment of maximum depolarization. Indifferent electrode in point R.

FIG. 2 Electrical circuit for measurement of standard I, II, III leads.

FIG. 3 Electrical circuit for measurement of standard aVR, aVL, aVF leads.

FIG. 4 Complete equivalent scheme of measurement of 6 standard leads I, II . . . aVF.

FIG. 5 Exact diagram of voltages in 6-axes coordinate system.

FIG. 6 Fluctuations of electrical asymmetry of voltages' ellipse during depolarization of healthy heart.

FIG. 7 Fluctuations of electrical asymmetry of voltages' ellipse during depolarization of heart with pathology.

FIG. 8 Consistency of electrical asymmetry of voltages' ellipse for healthy heart.

FIG. 9 Consistency of electrical asymmetry of voltages' ellipse for heart with pathology.

FIG. 10 Position of coordinate axes of topological model on anatomic portrait of heart's epicardium.

FIG. 11 Structure of information topological model of heart.

FIG. 12 Flowchart of device for a realization of the methods

FIG. 13 Portrait of healthy heart.

FIG. 14 Portrait of heart with pathology.

FIG. 15 ECG signal evolution for hidden ischemic disease.

FIG. 16 Heart portrait's evolution for hidden ischemic disease.

FIG. 17 Heart portrait for mild myocardial infarction.

### DETAILED DESCRIPTION OF THE INVENTION

The purpose of the given patent application consists in construction of a new type of topological model of ECG signals of standard leads, which in comparison with topological model of the method—prototype (patent DE 199 33 277 A1) should provide essential enhancement of sensitivity and specificity of the diagnostics with the earliest signs of developing diseases.

To reach this purpose the calculation of electrical potentials of additional body surface points is carried out which can be interpreted, as new leads, additional to standard 12 leads. Usage of additional leads allows more precise specification of localization of changes and type of pathology.

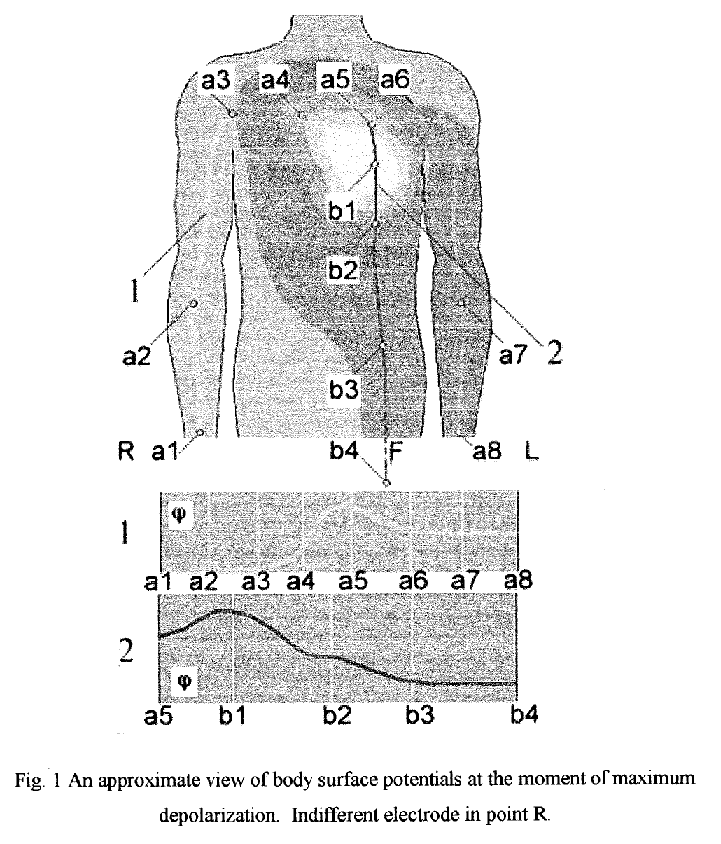

The essence of a method of definition of potentials of additional leads consists in the following. If the negative terminal of the measuring instrument to connect to a point R (right arm), the rough card of equipotent curves (lines of an identical potential) will remind a pattern represented on FIG. 1 (for the normal orientation of heart's electrical axis). If to conduct lines a1-a8 (line 1) and a5, b1-b4 (line 2), the change of a potential along these lines will look like the graphs 1 and 2 on FIG. 1. The parts a1-a3, a6-a8 and b3-b4 have practically constant potentials, i.e. these sites are passive conductors of an electric current.

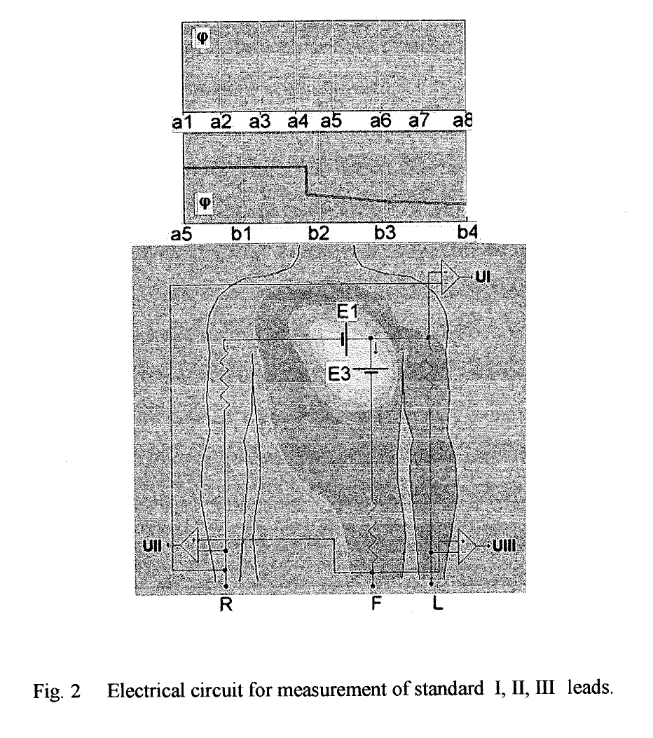

At the same time sites a4-a6 and a5-b2 have sources of electrical current. From the theory of electrical circuits it is known, that any potential diagram corresponds to some connection of passive units and sources of an electric current. The model simplification of the real potential diagrams FIG. 1 is represented in a top FIG. 2. Electric network at the bottom FIG. 2 corresponds to these models.

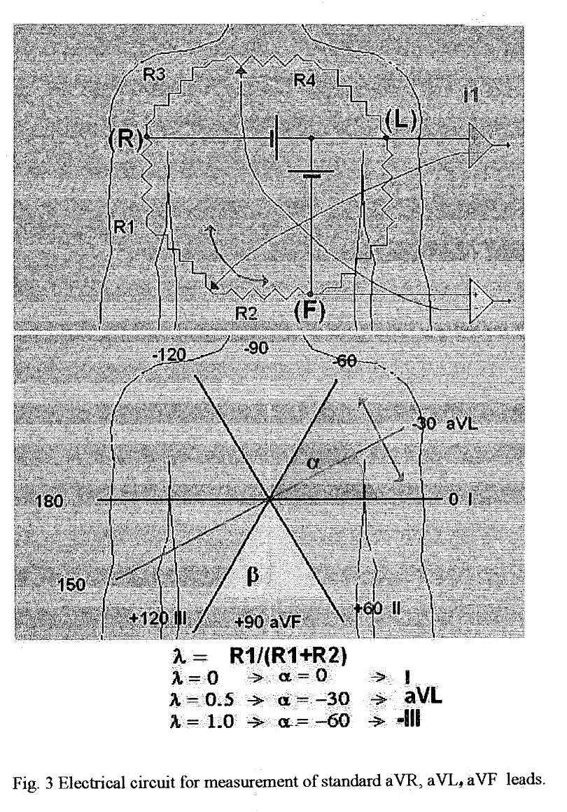

This circuit contains two current sources E1, E3, and three resistors. The resistors characterize conductivity of a human body between E1, E3 and points of connections of devices, which measure electrical potentials in leads I, II and III. Obviously, that voltage U1 of lead I is closed to the electromotive force (EMF) of a source E1, and U3 of lead III is closed to −EMF of a source E3. Because practically there is no voltage drop on resistors, this circuit for measurement of terminal potentials can be modified at FIG. 3. So we obtain a ring conductor passing through points of 3 standard leads I, II, III, and two EMF sources inside this ring.

Let's consider the device I1, which plus connected to a point L, and the minus “moves” on resistors between points R and F. When the minus paired to a point R, the instrument I1 measures lead I. When the minus paired to a point F, the instrument measures lead−U3. When the minus is in the middle between R and F (R1 equally R2), it measures lead aVL. If the minus of the device is in any intermediate point between R and F, we obtain some new lead in a six-axial coordinate system between leads I and III (see FIG. 3 bottom, gray sector). Let's enter the parameter λ=R1/(R1+R2), where R1 and R2 are resistances between current position of “minus” of device and points R and L. At change of a position of λa minus of the device this parameter varies from zero in a point R up to “1” in a point F. Accordingly corner in the standard cardiac 6-axes coordinate system varies from a minus 60 up to 0. Thus, value λ=0.5 corresponds to “reinforced” lead aVL (FIG. 3, bottom).

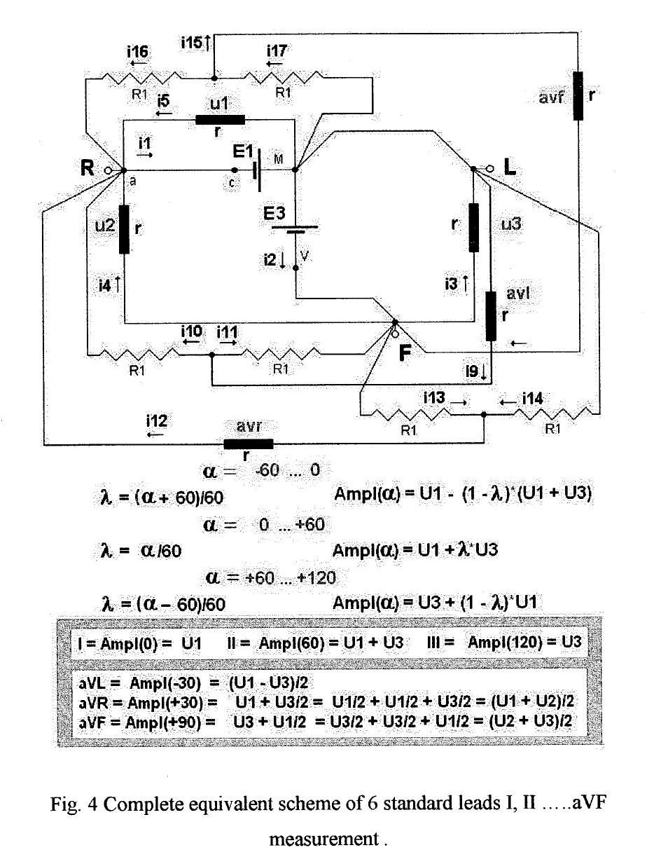

The similar correspondence exists and for sectors bound with leads aVF and aVR. For aVF this correspondence also is figured on FIG. 3. In the total, we obtain the circuit figured in a top FIG. 4. In this circuit the value r is an input resistance of the measuring instrument, and R1—part of the ring conductor. For certainty the “slipping” contact on FIG. 4 is placed in a middle position corresponding to leads aVR, aVL, aVF. Using Kirchgoff's laws after complex calculations of nodes and circuits, we receive the next relations:

If α=−60 . . . 0, then

λ=(α+60)/60 (f1)

Ampl (α, t)=U1 (t)−(1−λ)*(U1 (t)+U3(t)); (f2)

If a=0 . . . +60,

λ=α/60 (f3)

Ampl (α, t)=U1 (t)+λ*U3 (t); (f4)

If α=+60 . . . +λ20,

λ=(α−60)/60 (f5)

Ampl (α, t)=U3 (t)+(1−λ))*U1 (t); (f6)

(*—operation of multiplying).

The calculation is carried on in three sectors by 60 degrees. Input data are the voltage U1, U3 and parameter λ. It is possible to receive values U1, U3 at measurement of any combination of two leads from three standard I, II, III. If measure I and III,

U1=I, U3=III.

If measure I and II,

U1=I, U3=II−I.

If measure II and III,

U1=II−III, U3=III.

Thus, at any moment of time, measuring voltages only in two limb leads, i.e. I and III or I and II or II and III, it is possible to receive the extended field of signals for all intermediate angles of the 6 axial coordinate system. The relations for six standard terminal leads can be received at values of a corner 0 (I), +60 (II), +120 (III), +30 (aVR), −30 (aVL), +90 (aVF):

I=Ampl (0, t)=U1 (t) (f7)

II=Ampl (+60, t)=U1 (t)+U3 (t) (f8)

III=Ampl (+120, t)=U3 (t) (t9)

aVL=Ampl (+30, t)=U1 (t)/2=U3 (t)/2=(I−III)/2 (f10)

aVR=Ampl (+30, t)=U1 (t)+U3 (t)/2=(I+II)/2 (f11)

aVF=Ampl (+90, t)=U3 (t)+U1 (t)/2=(II+III)/2 (f12)

These relations are identical to what are utilized in ECG-devices, i.e. correspond to the outcomes implying from a vector dipole interpretation of average vector QRS generally accepted in heart electro physiology. However, in comparison with vector model values of potential differences essentially change for “intermediate” points, which corners are not matched to reinforced leads. In particular, became understandable known and rather disputable “mismatch” at 0.87 between a projection of an average vector QRS on 30 degrees turned axis and true value of a signal on this axis disappear in our approach.

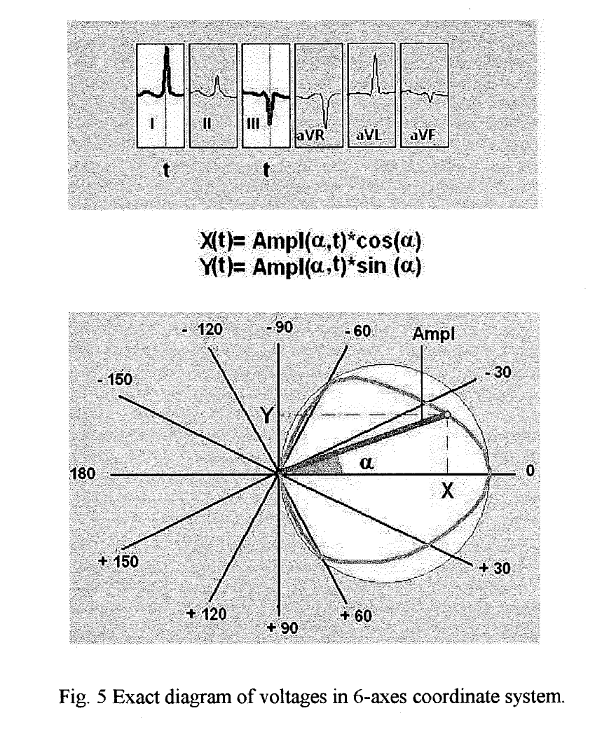

The process of the calculation is represented on FIG. 5. At first for each value of a corner α under the formulas FIG. 4 the value of Ampl (α) is determined. Further, on the value Ampl (α) calculate coordinates X, Y in a rectangular coordinate system under the formulas:

X=Ampl (α)*cos (α),

Y=Ampl (α)*sin (α) (*—operation of multiplying).

Further create a curve of change of voltage amplitude for any corner of a coordinate system (FIG. 5). This line can differ from an ideal circle corresponding a dipole model of electrical excitation of heart. And than more pathological deviation, the more difference of the ellipse QRS from a circle.

In the total, in each instant we obtain not only points corresponding terminal leads, but actually continuous line having, for example, 180 points(in this case one point corresponds to one degree of a coordinate system).. Thus, using only I and III leads we obtain the essential extension of the initial digital array on the basis of the improvements of the analysis of surface potentials. These extended arrays allow a detailed analysis of “QRS ellipse” changes in time.

Instead of an “average corner QRS” now it is possible to define in each instant “an instant corner (axis) QRS”. The instant axis QRS corresponds to a corner a, at which Ampl (α) achieves the maximum value. Thus all points, which are to the left of an instant axis QRS, correlate with changes in a left ventricle, and the points to the right of an axis QRS correlate with changes in a right ventricle.

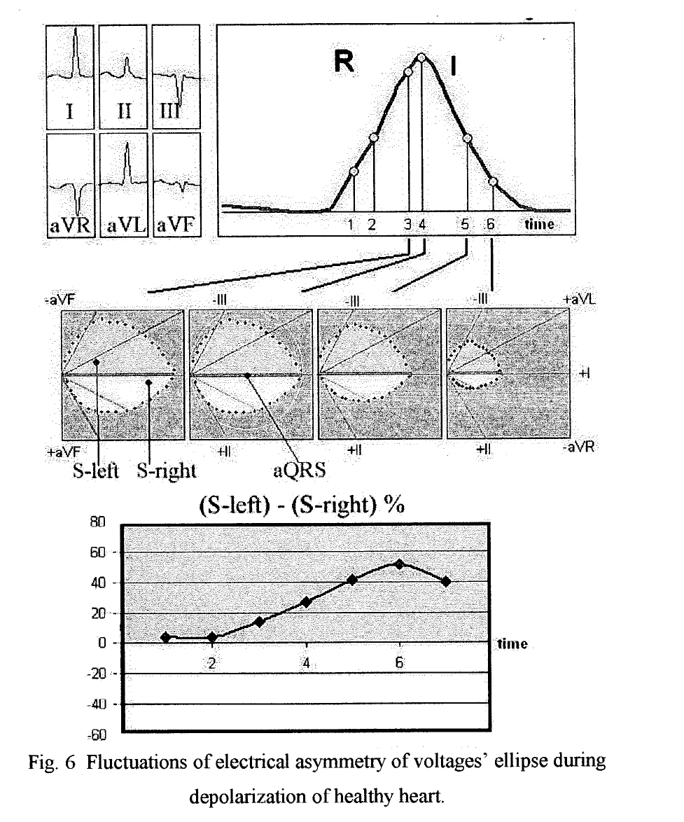

On FIG. 6 this ellipse evolution are represented during depolarization of ventricles of heart of the healthy man. The character of this evolution considerably varies at the very first signs of a myocardial infarction, when on the initial ECG still there are no changes. Similarly, for diagnostics of blockade of ventricles the large significance has change of an integral metric of electrical asymmetry of excitation of ventricles, which is a difference between squares of the ellipse to the left of a maximum and to the right of a maximum (bottom FIG. 6). Actually, we obtain the new diagnostic information on a synchronism of excitation of ventricles.

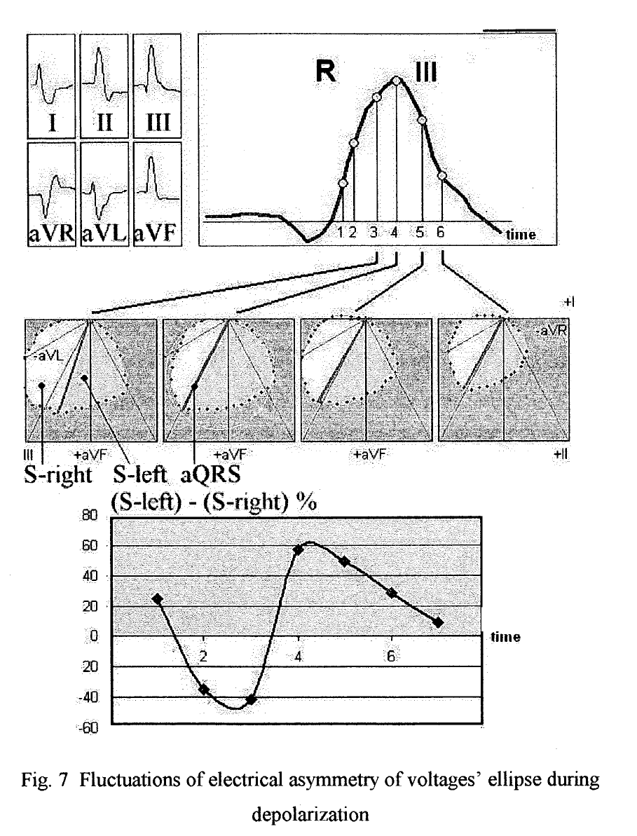

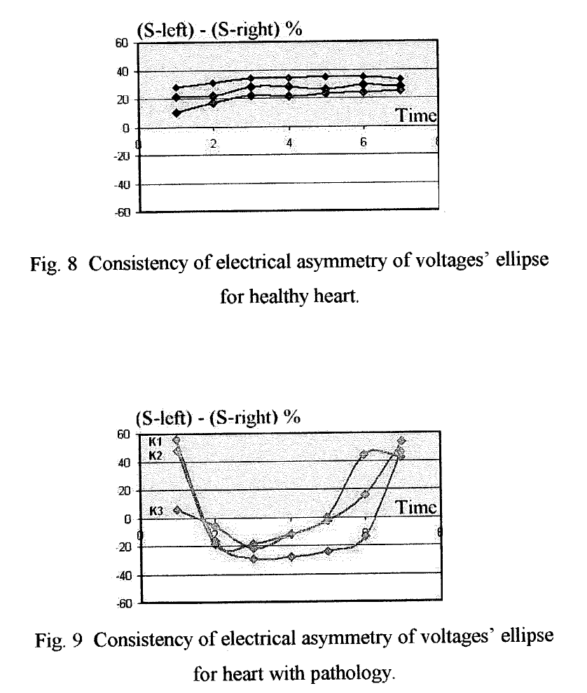

On FIG. 7 the similar information for heart of the man with a serious pathology represented. The considerable instability of the ellipse and completely other character of electrical asymmetry of depolarization of ventricles in matching with FIG. 6 are visible. The index of electrical asymmetry probably has high specificity, as it saves high recurrence for one patient. On FIG. 8 three sequential measurements of electrical asymmetry of ventricles for the patient with normal heart are showed, and on FIG. 9 three measurements for the patient with the large deviations are showed. The axis of time in these measurements corresponds to an interval between a beginning and the termination of wave R.

As opposed to the method - prototype, which operates with a data array of only 12 leads, in the given method up to 180 similar arrays are used which correspond to formal “intermediate” leads. Such considerable increase of the information makes possible to use as a base topological surface an anatomic model of a surface of heart.

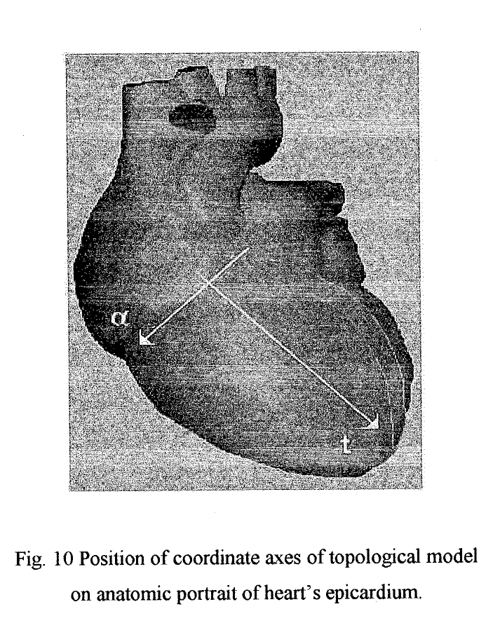

The layout of points of topological model is those, that the axis QRS approximately coincides with a projection of an inter ventricular septum, the corners a to the right from an axis QRS cover a surface of a right ventricle, and the corners α to the left from an axis QRS cover a surface of a left ventricle. There a direction from the basis of heart to its top corresponds to an axis of time (FIG. 10). Further, applying to each “intermediate” lead the procedure of a method prototype, we define secondary color and space coordinates, i.e. we cover with the certain color and space relief of a surface of the left and right ventricles of base topological model.

All circumscribed operations concern to the process of depolarization of ventricles, i.e. to wave R. However, the completely same operations are fair and for waves P (depolarization of atriums) and T (repolarization of ventricles).

Thus, the given method includes the following operations:

1. Specification of duration of ECG registration.

2. Selection of intervals of the analysis corresponding to separate PQRST-complex and clocked rather of a beginning wave R inside registered ECG.

3. Registration inside each interval of the analysis a voltage of any two standard leads: I-III, or I-II, or II-III. Define two base voltages U1 (t), U3 (t) under the following formulas:

If register I-III:

U1 (t)=I, U3 (t)=III.

If register I-II:

U1 (t)=I, U3 (t)=II-I.

If register II-III:

U1 (t)=II-III, U3 (t)=III.

4. Transformation of the U1 (t), U3 (t) data to digits.

5. Calculation the digital arrays of “intermediate” leads Ampl (α, t) (α-the corner in a six-axial coordinate system varies from −60 up to +120) under the following formulas:

If α=−60 . . . 0,

Ampl (α, t)=U1 (t)−(1−(α+60)/60)*(U1 (t)+U3 (t));

If α=0 . . . +60,

Ampl (α, t)=U1 (t)+(α/60)*U3 (t);

If α=+60 . . . +120,

Ampl (α, t)=U3 (t)+(1−(α−60)/60)*U1 (t);

(*—operation of multiplying).

The number of such arrays for practical implementation is defined by step of calculations on argument α. For at least step 1, this number is equal 180. Than less step on argument α, the more resolution of topological model.

6. For each stage of electrical excitation of heart, namely—for wave P, wave R and wave T the direction of an axis of maximum excitation is defined with the arrays Ampl (α, t). For this purpose in a point tmax, which corresponds to maximum amplitude of an appropriate wave, a corner β is defined, at which the value Ampl (β, tmax) has maximum value. The value divides the arrays Ampl (α, t) into the left and right halves. If a corner α<β, the array Ampl (α, t) correlates with the left departments of heart. If α>β, the array Ampl (α, t) correlates with the right departments of heart. In outcome, for each of considered waves receive three directions of maximum excitation—β_P for wave P, β_R for wave R, β_T for wave T.

7. Filtration of each array Ampl (α, t) through a high-pass filter, calculation of a difference between unfiltered and filtered signals, and representation of the obtained data according to the method—prototype, i.e. color encoding of a degree of deviation from standard given of healthy hearts obtained from clinic.

8. Filtration of the array Ampl (α, t) through a high-pass filter, calculation a difference between unfiltered and filtered signals, and usage of the obtained data according to the method—prototype for calculation of secondary space coordinates. Thus, function F_X (Ampl (α, t)), F_Y (Ampl (α, t)), F_Z (Ampl (α, t)), which specify secondary space codes:

Xr*=Xr+F_{—}X (Ampl (α, t))

Yr*=Yr+F_{—}Y (Ampl (α, t))

Zr*=Zr+F_{—}Z (Ampl (α, t)),

are selected so that for α<β coordinates Xr*, Yr* and Zr* are corresponding to the left departments of topological model, and under condition of α>β—right departments of topological model. Because the value β varies during electrical excitation, all effects of the enlarged (abnormal) electrical asymmetry of the left and right departments of heart are well visible on topological model.

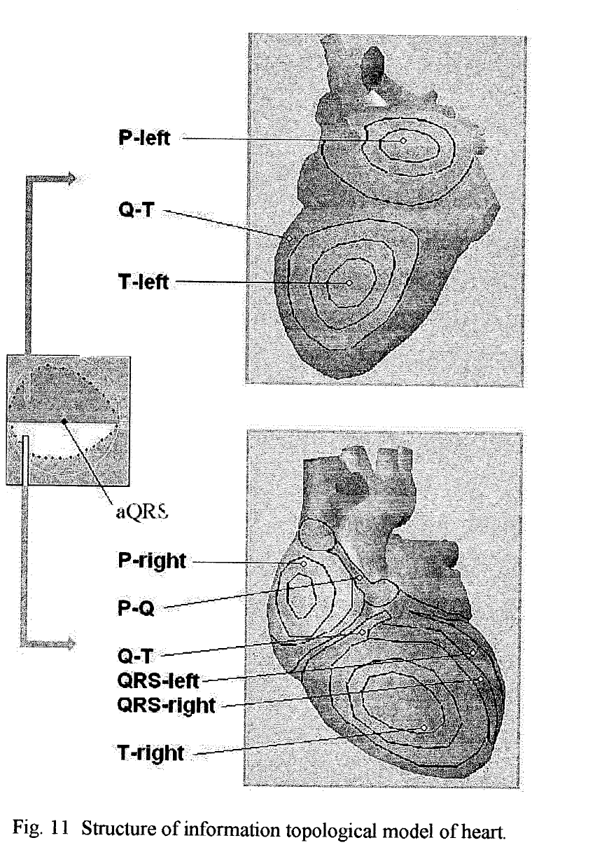

As a result the topological information model of heart is received, which structure is represented on FIG. 11. The model maps not only information on topology and amplitudes of electrical excitation for two atriums and two ventricles, but also contains the indirect data of the dynamics (dynamics of intervals P-Q and Q-T). The character of the color-coding corresponds to standards, which are used in human engineering: green or light-blue color corresponds to norm. Red parts of a color spectrum correspond to the large deviations.

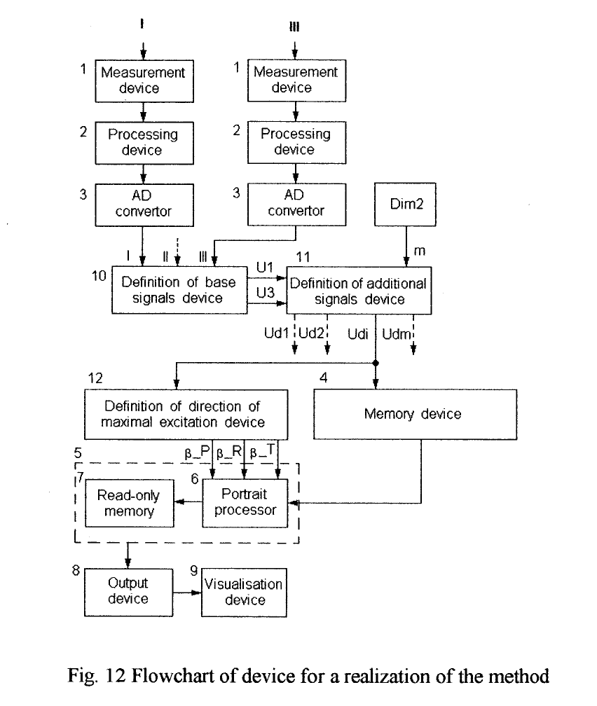

The flowchart of the device realizing the given method is represented on FIG. 12. Two lines of blocks (1,2,3) transform electrical voltages of two standard leads (for a certainty on FIG. 12 are selected I and III) in digital codes inside selected registration time according to procedures of the method—prototype for 12 leads. The output digital codes of two indicated lines of signals processing are transmitted to inputs I and III of the block (10)—“Devices of definition of base signals” (“Definition of base signals device”). The block (10) forms for any moment of time two signals U1 (t), U3 (t) in correspondence with algorithms of point 4. The signals U1 (t), U3 (t) are transferred on an input of the block (11)—“Devices of definition of additional signals” (“Definition of additional signals device”), which forms signals of additional leads Ud1 (t), . . . , Udm (t). The number of additional leads m (index of resolution of the analysis) is defined by a constant signal, which is transferred on an additional input of the block (11) from the block Dim2 and defines step of the analysis on the value of a corner a of the coordinate system. The step of 30 degrees (m=6) corresponds to the least resolution, the value of step of 1 degree (m=180) corresponds to the highest resolution. From an output of the block (11) arrays Udi (t), which number m is defined by a signal from the block Dim2, sequentially are transferred in the block (4) for further processing. The processing in blocks 4-9 corresponds to procedures of the method—prototype. The unique difference consists that three additional signals β_P, β_R, β_T from the block (12)—“Devices of direction finding of maximum excitation” (“Definition of direction of maximal excitation device”), are transmitted to an input of the block 6 (processor of a portrait). They are defining axes of maximum excitation for processes of depolarization of atriums, depolarization of ventricles and repolarization of ventricles. These signals are used in the block 3-2 (FIG.7 in the description of the method—prototype) for separation of the arrays of topological model on left and right according to operation 7 of the given description.

As a result of implementation of all circumscribed procedures we obtain a new method, which provides a building of information topological model of electrical excitation of heart in the form of anatomic portrait of heart. It gives the exact information on type and localization of pathology. This possibility can be provided only with new blocks 10, 11, 12 on FIG. 12, as these blocks allow calculating a direction of an axis of maximum excitation and distribution of amplitudes on both sides from this axis.

The information anatomic portrait of heart qualitatively changes physicians' operations, as instead of tiresome and the durable logical analysis of differential signs of ECG gives a possibility at once to see all picture of state changes as a whole. Thus the information portrait as against complete maps of potentials, gives not only anatomic topology of changes, but also information on the dynamics of operation of heart. Important advantage, that the color on an information portrait characterizes deviance from norm, instead of value of a potential, as at body surface mapping. p The secondary space of fluctuation features in the given method is considerably steadier in matching with space of standard differential signs of ECG analysis. It allows creating the very reliable automatic interpreter of heart portraits considerably exceeding on reliability of the automatic diagnosis existing interpreters of an ECG signal.

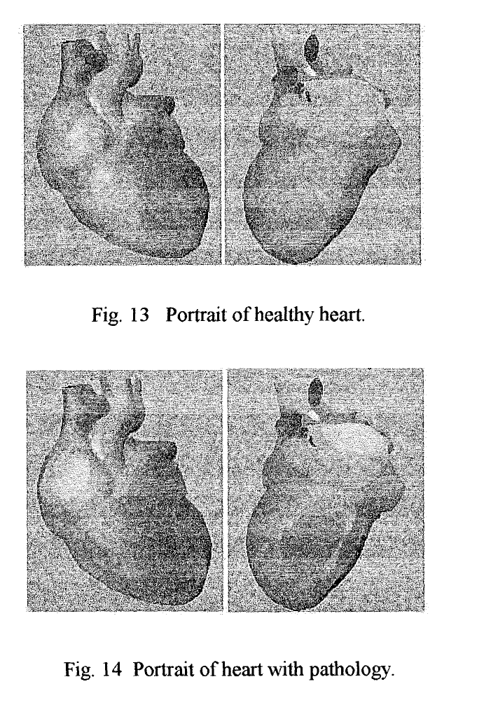

The examples of images obtained with the help of this method are represented on FIG. 13, FIG. 14. Time of input of an ECG-signal—30 sec. On FIG. 13 the heart portrait of the “healthy person” is represented in two projections—right-side view, left-side view. On FIG. 14—portrait of heart with a pathology. The obtained portraits have very high sensitivity and specificity, and also demonstrate very high recurrence of a portrait at a stable state of heart.

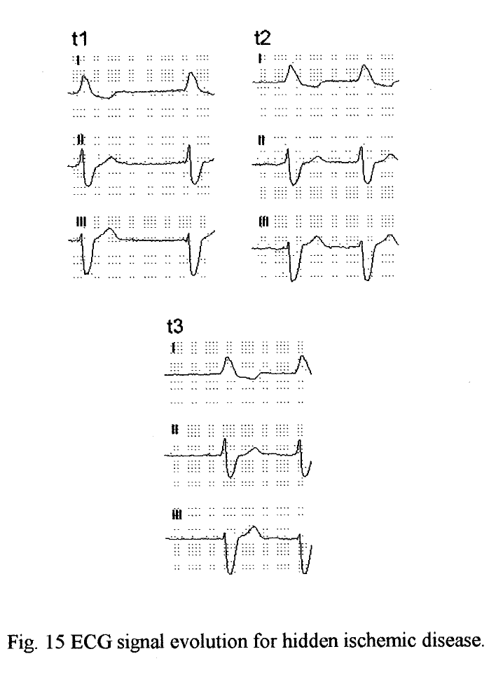

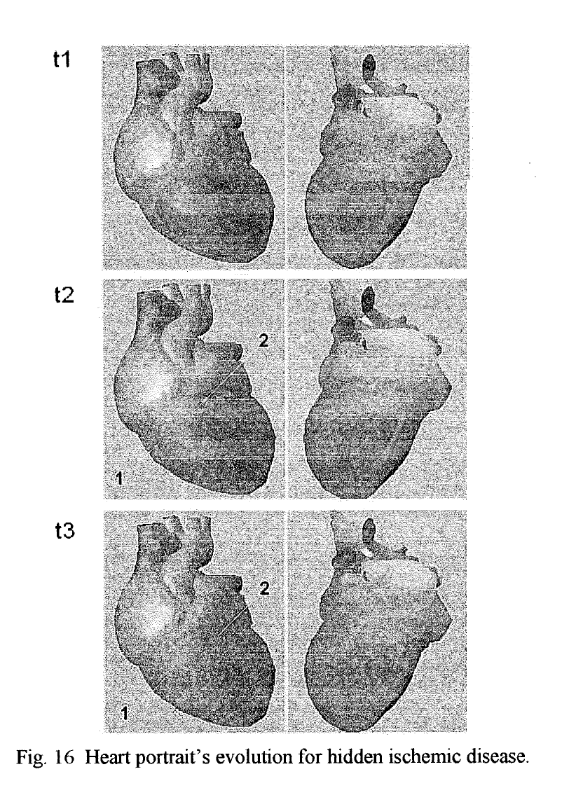

The illustrative example of sensitivity of a method is represented on FIG. 15, FIG. 16. Three examinations t1, t2, t3 are obtained during treatment of the patient in hospital with an interval in some days. The diagnosis is ischemic disease of heart, arteriosclerosis of coronary arteries. On FIG. 16 the entry ECG-signals I, II, III of leads, on FIG. 16—appropriate portraits of heart represented. On an ECG-signal it is difficult to see significant changes in these three examinations. At the same time the tendency of decrease of deviations in a right ventricle is well visible with heart portraits: the green color was partially restored in the field of 2, intensity of red color in “ellipse” 1 decreases noticeably and monotonically. These changes are reliable sign of slow improvement of a state, which is not visible with the traditional ECG analysis. The given example is a case history of unique possibilities of this device in the task of precise and operative observation of heart responses to executable therapy that is very important at choice and adjustment of tactics of medical treatment.

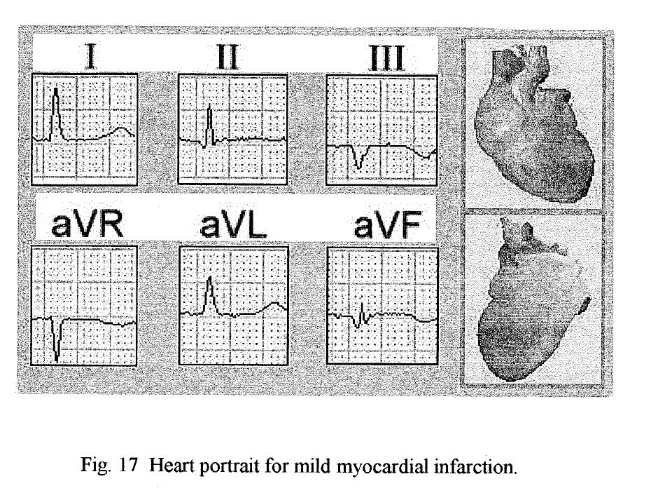

The example of sensitivity of the given method at detection of the not expressed pathologies is illustrated by diagnostics of a small local myocardial infarction on FIG. 17. The initial ECG signal does not contain essential signs of cicatricle changes—such electrocardiogram of terminal leads I—aVF in many cases corresponds to norm. However heart portrait, which is formed according to our method, has pathological view (red in the field of the left and right ventricles with prevalence in the side of a left ventricle) The computer interpreter recognizing heart portraits, in this case forms the following automatic conclusion “signs of cicatricle changes of lower localization, the complete clinical examination is necessary>> that corresponds to a true state.

What we claim as our invention is:

1. The method of visualization of ECG-signals of standard cardiological leads in the form of anatomic portrait of heart characterized by, that the data of any two standard leads I and III, I and II or II and III are transformed to the data of additional leads, which turn to a graphics portrait of heart in the form of anatomic model with distinguishing of four cameras of heart, color and form of which are determined by a state of heart.

2. The method of claim 1, characterized by, that
the time of the registration of the ECG-data is determined (time of exposure),
inside of the cycle of analysis the intervals of separate PQRST-complexes are selected,
inside each interval the data of any two from standard leads are transformed to digits,
the obtained data are transformed to the data of additional leads,
the colors are assigned to the data of additional leads, as the function of these data,
from the data of additional leads the space coordinates of base points of anatomic model with selection of axes of maximum electrical excitation of a myocardium are determined,
calculated color and space characteristics of a graphics image are transmitted in the data output device (device of visualization).

3. The method of claim 1 or 2, characterized by, that a beginning of each separate interval of the analysis synchronized with the beginning of the wave R, and termination of an interval of the analysis synchronized with the termination of wave T.

4. The method of at least one of the previous claims, characterized by, that the digital data of initial standard leads are transformed to digital data of additional leads.

5. The method of claim 4 characterized by, that number of additional leads can vary depending on necessary accuracy of localization of deviations (necessary resolution).

6. The method of claims 4 and 5, characterized by, that the additional leads are divided into group of leads posed to the left of an axis of maximum electrical excitation, and group of leads posed to the right of an axis of maximum electrical excitation.

7. The method of at least one of the previous claims characterized by, that the digital data of each additional lead on one interval of the registration are passed through a high-pass filter, then a difference between unfiltered and filtered signals is determine, and the obtained data are encoded by color on a degree of deviation from standard data of healthy heart obtained empirically.

8. The method of at least one of the previous claims characterized by, that the space coordinates of base points of additional leads posed to the left of an axis of maximum excitation, correspond to the left departments of heart, and the space coordinates of additional leads to the right of an axis of maximum excitation correspond to the right departments of heart.

9. The method of at least by one of the previous claims characterized by, that an anatomic portrait of heart is supplemented by the conclusions of the portraits' interpreter, realizing automatic classification of portraits of heart. The device for realization of a method of claim 1, including:
device (1) for registration of the ECG-data and transformation them in electrical signals,
device (2) for definition of interval of the registration,
AD-converter (3),
processor of a portrait (6),
storage device (7) with the program of the analysis and standard data base,
output device (8),
video screen monitor (9)
characterized by the devices of conversion (10,11,12) with the analytical software intended for definition of data of additional leads and a direction of an axis of maximum electrical excitation.

11. The device of claim 10, characterized by, that the device (10) transforms voltages of any combination of two standard leads I-II, I-III or II-III to two basic voltages.

12. The device of claim 10, characterized by, that the device (11) will transform two basic voltages to outputting additional voltages, which number is defined by the value of a constant signal of localization accuracy transmitted from the switch Dim2.

13. The device of claim 10, characterized by, that the device (12) will transform outputting additional voltages to three signals of corners of maximum excitation accordingly for processes of depolarization of atriums, depolarization of ventricles and repolarization of ventricles.

14. The device of claim 10, characterized by, that the output signals of the device (12) are transferred in the device (6) for handle of space localization of deviations on an anatomic portrait of heart.
